(19) United States
(12) Patent Application Publication
(10) Pub. No.: US 2024/0352539 A1
(43) Pub. Date: Oct. 24, 2024

(54) PATHOGEN SPECIFIC NUCLEIC ACID FRAGMENT AND APPLICATION THEREOF

(71) Applicant: METHYCURE BIOTECH CORP., Jiangsu (CN)

(72) Inventors: Qihan Chen, Jiangsu (CN); Qiang Wang, Jiangsu (CN)

(21) Appl. No.: 18/245,619

(22) PCT Filed: Sep. 16, 2021
(86) PCT No.: PCT/CN2021/118722

§ 371 Date: Mar. 16, 2023

(30) Foreign Application Priority Data
Sep. 16, 2020 (WO) PCT/CN2020/115682

(51) Int. Cl. C12Q 1/689

(52) U.S. Cl. CPC C12Q 1/689

(57) ABSTRACT

Provided are a plurality of pathogen specific nucleic acid fragments. Also provided is a method for identifying one or more pathogens, comprising: 1) providing a sample that may comprise the pathogens; and 2) detecting whether a pathogen specific nucleic acid fragment exits in the sample or not or detecting the content of pathogen specific nucleic acid fragment in the sample, wherein the presence or absence of the pathogen specific nucleic acid fragment or the content thereof respectively reflects the presence or absence of a pathogen corresponding to the pathogen specific nucleic acid fragment in the sample or the content thereof. The provided pathogen specific nucleic acid fragment can be applied to rapid identification of pathogens, and in clinical application, the positive detection rate is high and the detection period is short.

### CROSS-REFERENCE TO RELATED APPLICATION

This application claims the benefit of priority to International Patent Application No. PCT/CN2020/115682 filed Sep. 16, 2020, the disclosure of which is incorporated herein in its entirety by reference.

### REFERENCE TO SEQUENCE LISTING IN COMPUTER READABLE FORMAT

An electronic sequence listing in computer readable format (file name: P10886PCT.210916.SEQUENCE LISTING_ST25.txt, filed Sep. 16, 2021, size: 1,011 KB) is filed with this application, the disclosure of which is incorporated herein in its entirety by reference.

### TECHNICAL FIELD

The present invention relates to a pathogen specific nucleic acid fragment, and also relates to a method for utilizing the pathogen specific nucleic acid fragment for identification of a pathogen in a sample.

### BACKGROUND

The identification of pathogenic bacteria from patient samples based on colony culture is a traditional pathogen detection method and currently a common method in the clinic. This method requires aseptically drawing a patient's blood or aseptically collecting a patient's body fluid sample, transferring it to a sterile culture bottle for pathogenic bacteria enrichment culture, subsequently seeding it to a plate culture dish, and finally picking a single clone for chromoscopic observation. By performing differential growth experiments on selective media or performing various biochemical tests, the identification of pathogenic bacteria in the patient's sample is achieved according to the phenotypic and physiological characteristics of the pathogenic bacteria. This assay is not only time-consuming (usually requires one to several days of colony enrichment culture), but also lead to negative results because of the harsh culture conditions for some anaerobic pathogenic bacteria or other strains hard to be cultured in common conditions. Meanwhile, contamination may be introduced during the process of bacterial culture and isolation, resulting in false-positive test results, which may lead to misdiagnosis and the misuse of antimicrobial drugs.

Pneumonia, a disease resulting from invasion and overgrowth of pathogens in the soft tissues of the lungs, can cause unilateral or bilateral lung alveolar inflammation, with the respiratory tract filled with fluid or pus, which in turn triggers dyspnea. It is defined by the World Health Organization (WHO) as an acute respiratory infection affecting the soft tissues and oxygenation of the lungs, and its clinical diagnosis is based on the presence of shadowing on chest X-ray. Currently, pneumonia remains a serious disease that seriously affects public health, with pneumonia accounting for more deaths than any other infectious disease in the United States according to a 2016 mortality trend analysis, and without any improvement during the previous 34 years. Worldwide, the morbidity and mortality of neonatal pneumonia is persistently high, and 152000-490000 infants under the age of one year die from pneumonia every year, making it a veritable “child killer”. Severe pneumonia is a progressive lung inflammation, caused by pulmonary infection leading to a systemic inflammatory response, with the development of the disease worsening, even causing systemic severe infection diseases such as respiratory failure, septic shock, sepsis. Severe pneumonia is defined by the WHO as a patient having cough or dyspnea and symptoms such as lower chest wall adduction or vomiting, impaired consciousness, central cyanosis, or peripheral oxygen saturation less than 90%. Despite the rapid development of antibiotic therapy and life support therapy in recent years, severe pneumonia remains one of the leading causes of intensive care unit admission and death. There are a large variety of pathogenic bacteria causing severe pneumonia, and the resistance of various pathogenic bacteria to different antibiotics is quite different. Current detection technology of clinical pathogenic bacteria is still far from meeting the need for clinical diagnosis and treatment of patients with severe pneumonia.

### SUMMARY

In one aspect, the present invention provides a method for identifying one or more pathogens, comprising:
(1) providing a sample that may comprise the pathogen; and
(2) detecting presence or absence of a pathogen specific nucleic acid fragment in the sample or detecting content of a pathogen specific nucleic acid fragments in the sample,
wherein a pathogen specific nucleic acid fragment corresponding to Acinetobacter baumannii is selected from at least a part of any sequence of SEQ ID No: 34-49, or a complementary sequence thereof;
a pathogen specific nucleic acid fragment corresponding to Escherichia coli is selected from at least a part of any sequence of SEQ ID No: 50-221, or a complementary sequence thereof;
a pathogen specific nucleic acid fragment corresponding to Klebsiella pneumoniae is selected from at least a part of any sequence of SEQ ID No: 222-542, or a complementary sequence thereof;
a pathogen specific nucleic acid fragment corresponding to Staphylococcus aureus is selected from at least a part of any sequence of SEQ ID No: 543-601, or a complementary sequence thereof;
a pathogen specific nucleic acid fragment corresponding to Pseudomonas aeruginosa is selected from at least a part of any sequence of SEQ ID No: 602-896, or a complementary sequence thereof;
a pathogen specific nucleic acid fragment corresponding to Staphylococcus epidermidis is selected from at least a part of any sequence of SEQ ID No: 897-1079, or a complementary sequence thereof;
a pathogen specific nucleic acid fragment corresponding to Staphylococcus capitis is selected from at least a part of any sequence of SEQ ID No: 1080-1169, or a complementary sequence thereof;
a pathogen specific nucleic acid fragment corresponding to Enterococcus faecalis is selected from at least a part of any sequence of SEQ ID No: 1170-1279, or a complementary sequence thereof;
a pathogen specific nucleic acid fragment corresponding to Enterococcus faecium is selected from at least a part of any sequence of SEQ ID No: 1280-1405, or a complementary sequence thereof;
a pathogen specific nucleic acid fragment corresponding to Stenotrophomonas maltophilia is selected from at least a part of any sequence of SEQ ID No: 1406-1550, or a complementary sequence thereof;
a pathogen specific nucleic acid fragment corresponding to Mycobacterium tuberculosis, Mycobacterium africanum, or Mycobacterium bovis is selected from at least a part of SEQ ID No: 1551-1590, or a complementary sequence thereof,
wherein the presence or content of the pathogen specific nucleic acid fragment reflects the presence or content of the pathogen corresponding to the pathogen-specific nucleic acid fragment in the sample, respectively.

In some embodiments, the step (2) further comprises performing extraction of nucleic acid from the sample prior to the detection.

In some embodiments, the step (2) comprises performing an amplification reaction using the pathogen specific nucleic acid fragment in the sample as a template, and determining the presence or content of the pathogen-specific nucleic acid fragment by detecting presence or content of amplified product.

In some embodiments, the amplification reaction is Polymerase Chain Reaction (PCR).

In some embodiments, in the PCR: primers used for amplification of pathogen specific nucleic acid fragment of Acinetobacter baumannii comprise sequences as set forth in SEQ ID No: 1 and SEQ ID No: 2; primers used for amplification of pathogen specific nucleic acid fragment of Klebsiella pneumoniae comprise sequences as set forth in SEQ ID No: 4 and SEQ ID No: 5; primers used for amplification of pathogen specific nucleic acid fragment of Staphylococcus aureus comprise sequences as set forth in SEQ ID No: 7 and SEQ ID No: 8; primers used for amplification of pathogen specific nucleic acid fragment of Escherichia coli comprise sequences as set forth in SEQ ID No: 10 and SEQ ID No: 11; primers used for amplification of pathogen specific nucleic acid fragment of Pseudomonas aeruginosa comprise sequences as set forth in SEQ ID No: 13 and SEQ ID No: 14; primers used for amplification of pathogen specific nucleic acid fragment of Stenotrophomonas maltophilia comprise sequences as set forth in SEQ ID No: 16 and SEQ ID No: 17; primers used for amplification of pathogen specific nucleic acid fragment of Staphylococcus epidermidis comprise sequences as set forth in SEQ ID No: 19 and SEQ ID No: 20; primers used for amplification of pathogen specific nucleic acid fragment of Enterococcus faecium comprise sequences as set forth in SEQ ID No: 22 and SEQ ID No: 23; primers used for amplification of pathogen specific nucleic acid fragment of Staphylococcus capitis comprise sequences as set forth in SEQ ID No: 25 and SEQ ID No: 26; primers used for amplification of pathogen specific nucleic acid fragment of Enterococcus faecalis comprise sequences as set forth in SEQ ID No: 28 and SEQ ID No: 29; primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32.

In some embodiments, the detection of amplification product is performed utilizing trans cleavage activity of a nuclease of CRISPR/Cas family.

In some embodiments, the nuclease of CRISPR/Cas family is Cas12.

In some embodiments, the nuclease of CRISPR/Cas family is lbCas12.

In some embodiments, the primers used for amplification of pathogen specific nucleic acid fragment of Acinetobacter baumannii comprise sequences as set forth in SEQ ID No: 1 and SEQ ID No: 2, and a target sequence of crRNA used in combination with the nuclease of CRISPR/Cas family comprises a sequence as set forth in SEQ ID No: 3; the primers used for amplification of pathogen specific nucleic acid fragment of Klebsiella pneumoniae comprise sequences as set forth in SEQ ID No: 4 and SEQ ID No: 5, and a target sequence of crRNA used in combination with the nuclease of CRISPR/Cas family comprises a sequence as set forth in SEQ ID No: 6; the primers used for amplification of pathogen specific nucleic acid fragment of Staphylococcus aureus comprise sequences as set forth in SEQ ID No: 7 and SEQ ID No: 8, and a target sequence of crRNA used in combination with the nuclease of CRISPR/Cas family comprises a sequence as set forth in SEQ ID No: 9; the primers used for amplification of pathogen specific nucleic acid fragment of Escherichia coli comprise sequences as set forth in SEQ ID No: 10 and SEQ ID No: 11, and a target sequence of crRNA used in combination with the nuclease of CRISPR/Cas family comprises a sequence as set forth in SEQ ID No: 12; the primers used for amplification of pathogen specific nucleic acid fragment of Pseudomonas aeruginosa comprise sequences as set forth in SEQ ID No: 13 and SEQ ID No: 14, and a target sequence of crRNA used in combination with the nuclease of CRISPR/Cas family comprises a sequence as set forth in SEQ ID No: 15;
the primers used for amplification of pathogen specific nucleic acid fragment of Stenotrophomonas maltophilia comprise sequences as set forth in SEQ ID No: 16 and SEQ ID No: 17, and a target sequence of crRNA used in combination with the nuclease of CRISPR/Cas family comprises a sequence as set forth in SEQ ID No: 18;
the primers used for amplification of pathogen specific nucleic acid fragment of Staphylococcus epidermidis comprise sequences as set forth in SEQ ID No: 19 and SEQ ID No: 20, and a target sequence of crRNA used in combination with the nuclease of CRISPR/Cas family comprises a sequence as set forth in SEQ ID No: 21;
the primers used for amplification of pathogen specific nucleic acid fragment of Enterococcus faecium comprise sequences as set forth in SEQ ID No: 22 and SEQ ID No: 23, and a target sequence of crRNA used in combination with the nuclease of CRISPR/Cas family comprises a sequence as set forth in SEQ ID No: 24;
the primers used for amplification of pathogen specific nucleic acid fragment of Staphylococcus capitis comprise sequences as set forth in SEQ ID No: 25 and SEQ ID No: 26, and a target sequence of crRNA used in combination with the nuclease of CRISPR/Cas family comprises a sequence as set forth in SEQ ID No: 27;
the primers used for amplification of pathogen specific nucleic acid fragment of Enterococcus faecalis comprise sequences as set forth in SEQ ID No: 28 and SEQ ID No: 29, and a target sequence of crRNA used in combination with the nuclease of CRISPR/Cas family comprises a sequence as set forth in SEQ ID No: 30;
the primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32, and a target sequence of crRNA used in combination with the nuclease of CRISPR/Cas family comprises a sequence as set forth in SEQ ID No: 33.

In some embodiments, the sample is sputum or alveolar lavage fluid from a patient with severe pneumonia.

In some embodiments, identification is performed in step (2) for a plurality of pathogens, including Acinetobacter baumannii, Escherichia coli, Klebsiella pneumoniae, Staphylococcus aureus, Pseudomonas aeruginosa, Staphylococcus epidermidis, Staphylococcus capitis, Enterococcus faecalis, Enterococcus faecium, and Stenotrophomonas maltophilia.

In some embodiments, the plurality of pathogens further comprise Mycobacterium tuberculosis, Mycobacterium africanum, or Mycobacterium bovis.

In another aspect, the present invention provides an isolated nucleic acid molecule comprising at least a part of any sequence of SEQ ID No: 34-1590, or a complementary sequence thereof.

In some embodiments, the nucleic acid molecule has a length of not less than 60 nucleotides.

In another aspect, the present invention provides a kit for detection of pathogen in a sample, comprising
(1) primers for amplification of pathogen specific nucleic acid fragments in the sample to generate amplification product; and
(2) a nuclease of CRISPR/Cas family with trans cleavage activity, a crRNA using at least a part of sequence of the amplification product as target sequence, and a single-stranded DNA reporter molecule with a fluorophore and a quencher group at the 5′ and 3′ ends, respectively, wherein
a pathogen specific nucleic acid fragment corresponding to Acinetobacter baumannii is selected from at least a part of any sequence of SEQ ID No: 34-49, or a complementary sequence thereof; a pathogen specific nucleic acid fragment corresponding to Escherichia coli is selected from at least a part of any sequence of SEQ ID No: 50-221, or a complementary sequence thereof; a pathogen specific nucleic acid fragment corresponding to Klebsiella pneumoniae is selected from at least a part of any sequence of SEQ ID No: 222-542, or a complementary sequence thereof; a pathogen specific nucleic acid fragment corresponding to Staphylococcus aureus is selected from at least a part of any sequence of SEQ ID No: 543-601, or a complementary sequence thereof, a pathogen specific nucleic acid fragment corresponding to Pseudomonas aeruginosa is selected from at least a part of any sequence of SEQ ID No: 602-896, or a complementary sequence thereof, a pathogen specific nucleic acid fragment corresponding to Staphylococcus epidermidis is selected from at least a part of any sequence of SEQ ID No: 897-1079, or a complementary sequence thereof, a pathogen specific nucleic acid fragment corresponding to Staphylococcus capitis is selected from at least a part of any sequence of SEQ ID No: 1080-1169, or a complementary sequence thereof, a pathogen specific nucleic acid fragment corresponding to Enterococcus faecalis is selected from at least a part of any sequence of SEQ ID No: 1170-1279, or a complementary sequence thereof, a pathogen specific nucleic acid fragment corresponding to Enterococcus faecium is selected from at least a part of any sequence of SEQ ID No: 1280-1405, or a complementary sequence thereof, a pathogen specific nucleic acid fragment corresponding to Stenotrophomonas maltophilia is selected from at least a part of any sequence of SEQ ID No: 1406-1550, or a complementary sequence thereof, and a pathogen specific nucleic acid fragment corresponding to Mycobacterium tuberculosis, Mycobacterium africanum, or Mycobacterium bovis is selected from at least a part of any sequence of SEQ ID No: 1551-1590, or a complementary sequence thereof.

In some embodiments, the kit further comprises the above nucleic acid fragment as a positive control.

In some embodiments, the nuclease of CRISPR/Cas family is lbCas12.

In some embodiments, the primers used for amplification of pathogen specific nucleic acid fragment of Acinetobacter baumannii comprise sequences as set forth in SEQ ID No: 1 and SEQ ID No: 2; the primers used for amplification of pathogen specific nucleic acid fragment of Klebsiella pneumoniae comprise sequences as set forth in SEQ ID No: 4 and SEQ ID No: 5; the primers used for amplification of pathogen specific nucleic acid fragment of Staphylococcus aureus comprise sequences as set forth in SEQ ID No: 7 and SEQ ID No: 8; the primers used for amplification of pathogen specific nucleic acid fragment of Escherichia coli comprise sequences as set forth in SEQ ID No: 10 and SEQ ID No: 11; the primers used for amplification of pathogen specific nucleic acid fragment of Pseudomonas aeruginosa comprise sequences as set forth in SEQ ID No: 13 and SEQ ID No: 14; the primers used for amplification of pathogen specific nucleic acid fragment of Stenotrophomonas maltophilia comprise sequences as set forth in SEQ ID No: 16 and SEQ ID No: 17; the primers used for amplification of pathogen specific nucleic acid fragment of Staphylococcus epidermidis comprise sequences as set forth in SEQ ID No: 19 and SEQ ID No: 20; the primers used for amplification of pathogen specific nucleic acid fragment of Enterococcus faecium comprise sequences as set forth in SEQ ID No: 22 and SEQ ID No: 23; the primers used for amplification of pathogen specific nucleic acid fragment of Staphylococcus capitis comprise sequences as set forth in SEQ ID No: 25 and SEQ ID No: 26; the primers used for amplification of pathogen specific nucleic acid fragment of Enterococcus faecalis comprise sequences as set forth in SEQ ID No: 28 and SEQ ID No: 29; the primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32.

In some embodiments, the primers used for amplification of pathogen specific nucleic acid fragment of Acinetobacter baumannii comprise sequences as set forth in SEQ ID No: 1 and SEQ ID No: 2, and a target sequence of the crRNA comprises a sequence as set forth in SEQ ID No: 3; the primers used for amplification of pathogen specific nucleic acid fragment of Klebsiella pneumoniae comprise sequences as set forth in SEQ ID No: 4 and SEQ ID No: 5, and a target sequence of the crRNA comprises a sequence as set forth in SEQ ID No: 6; the primers used for amplification of pathogen specific nucleic acid fragment of Staphylococcus aureus comprise sequences as set forth in SEQ ID No: 7 and SEQ ID No: 8, and a target sequence of the crRNA comprises a sequence as set forth in SEQ ID No: 9; the primers used for amplification of pathogen specific nucleic acid fragment of Escherichia coli comprise sequences as set forth in SEQ ID No: 10 and SEQ ID No: 11, and a target sequence of the crRNA comprises a sequence as set forth in SEQ ID No: 12; the primers used for amplification of pathogen specific nucleic acid fragment of Pseudomonas aeruginosa comprise sequences as set forth in SEQ ID No: 13 and SEQ ID No: 14, and a target sequence of the crRNA comprises a sequence as set forth in SEQ ID No: 15; the primers used for amplification of pathogen specific nucleic acid fragment of Stenotrophomonas maltophilia comprise sequences as set forth in SEQ ID No: 16 and SEQ ID No: 17, and a target sequence of the crRNA comprises a sequence as set forth in SEQ ID No: 18; the primers used for amplification of pathogen specific nucleic acid fragment of Staphylococcus epidermidis comprise sequences as set forth in SEQ ID No: 19 and SEQ ID No: 20, and a target sequence of the crRNA comprises a sequence as set forth in SEQ ID No: 21; the primers used for amplification of pathogen specific nucleic acid fragment of Enterococcus faecium comprise sequences as set forth in SEQ ID No: 22 and SEQ ID No: 23, and a target sequence of the crRNA comprises a sequence as set forth in SEQ ID No: 24; the primers used for amplification of pathogen specific nucleic acid fragment of Staphylococcus capitis comprise sequences as set forth in SEQ ID No: 25 and SEQ ID No: 26, and a target sequence of the crRNA comprises a sequence as set forth in SEQ ID No: 27; the primers used for amplification of pathogen specific nucleic acid fragment of Enterococcus faecalis comprise sequences as set forth in SEQ ID No: 28 and SEQ ID No: 29, and a target sequence of the crRNA comprises a sequence as set forth in SEQ ID No: 30; the primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32, and a target sequence of the crRNA comprises a sequence as set forth in SEQ ID No: 33.

In some embodiments, the sample is sputum or alveolar lavage fluid from a patient with severe pneumonia.

In some embodiments, the kit is used for detection of Acinetobacter baumannii, Escherichia coli, Klebsiella pneumoniae, Staphylococcus aureus, Pseudomonas aeruginosa, Staphylococcus epidermidis, Staphylococcus capitis, Enterococcus faecalis, Enterococcus faecium, and Stenotrophomonas maltophilia in the sample.

In some embodiments, the kits are used for detection of Acinetobacter baumannii, Escherichia coli, Klebsiella pneumoniae, Staphylococcus aureus, Pseudomonas aeruginosa, Staphylococcus epidermidis, Staphylococcus capitis, Enterococcus faecalis, Enterococcus faecium, and Stenotrophomonas maltophilia in the sample and Mycobacterium tuberculosis, Mycobacterium africanum, or Mycobacterium bovis in the sample.

The pathogen specific nucleic acid fragments presented herein can be used for rapid identification of pathogens with high positive detection rate and short detection period (e.g. less than 3 hours) in clinical application.

### DESCRIPTION OF DRAWINGS

FIG. 1 shows a flowchart of the method described herein for obtaining pathogen-specific nucleic acid fragments.

FIG. 2 shows a schematic diagram showing the trans cleavage activity of Cas12a.

FIG. 3 shows a specific nucleic acid fragment of Acinetobacter baumannii, with the position of corresponding primers, crRNA target sequence and PAM sequence indicated.

FIG. 4 shows the electrophoresis results of the amplified products after amplification of DNA templates from various pathogens with 10 pairs of amplification primers. M: 250 bp gradient ladder; Lanes 1-10 in turn indicate the genomic DNA templates amplified by PCR for each group as follows: Lane 1: Acinetobacter baumannii; Lane 2: Escherichia coli; Lane 3: Klebsiella pneumoniae; Lane 4: Staphylococcus aureus; Lane 5: Pseudomonas aeruginosa; Lane 6: Staphylococcus epidermidis; Lane 7: Staphylococcus capitis; Lane 8: Enterococcus faecalis; Lane 9: Enterococcus faecium; Lane 10: Stenotrophomonas maltophilia; NC was the negative control, i.e. the template was deionized water.

FIG. 5 shows the results of fluorescence signal detection of different amplification products with lbCas12a and crRNA. Each set of crRNA was used to detect the PCR products amplified by corresponding primers, and the horizontal ordinate 1-10 represented the amplified products from the following pathogens, respectively: Lane 1: Acinetobacter baumannii; Lane 2: Escherichia coli; Lane 3: Klebsiella pneumoniae; Lane 4: Staphylococcus aureus; Lane 5: Pseudomonas aeruginosa; Lane 6: Staphylococcus epidermidis; Lane 7: Staphylococcus capitis; Lane 8: Enterococcus faecalis; Lane 9: Enterococcus faecium; Lane 10: Stenotrophomonas maltophilia; NC was the negative control, i.e. the template for PCR was deionized water. Error bars represent the mean±S.E.M. of fluorescence signals, with 3 replicates per group.

FIG. 6 shows the results of the distribution of pathogenic bacteria identified using lbCas12a for the patient's clinical sample after nucleic acid amplification. PC: positive control, i.e., subjects with pathogenic bacterial genomic DNA; the heat map indicates the percentage of the mean fluorescence values for each group of reactions; the symbol #represents a positive result for the conventional culture method.

### DETAILED DESCRIPTION OF EMBODIMENTS

Unless otherwise stated, all technical and scientific terms as used herein have the meanings commonly understood by those of ordinary skill in the art.

### Pathogen Specific Nucleic Acid Fragments

“pathogen specific nucleic acid fragments”, also referred to as pathogenic bacteria or pathogen specific nucleic acid fragments (usually referring to DNA sequences and can also comprise RNA sequences), refer to: DNA sequences that are all widely present in the genomes of strains taxonomically belonging to the same pathogens, but that are not present in the genomes of other species of pathogens, that is, intra-specie consensus inter-specie specific DNA sequences. The species of bacteria can be clearly delineated on the basis of DNA sequences unique to pathogenic bacteria.

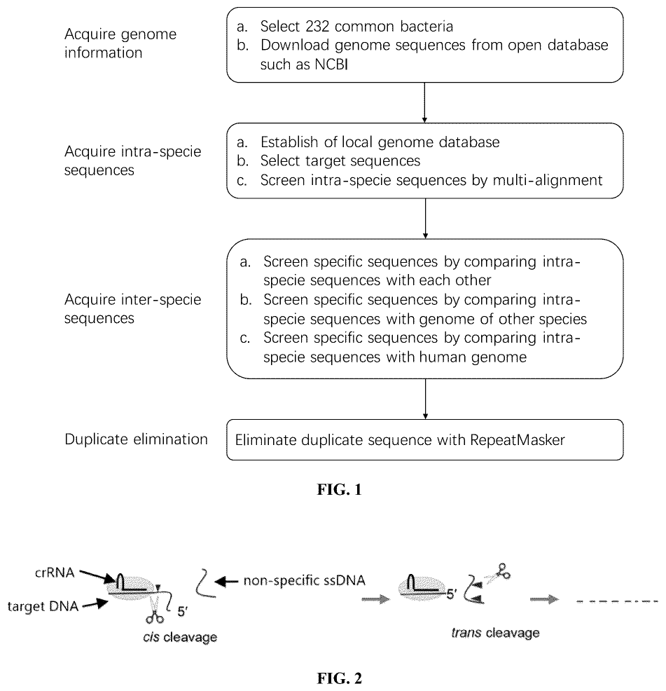

In some embodiments, DNA sequences specific to pathogenic bacteria are obtained herein by the following steps (see FIG. 1):
1) Obtaining whole genome sequences: whole genome sequences of hundreds of common microorganisms were obtained from public databases such as NCBI;
2) Acquisition of intraspecific consensus sequence of the same species of bacteria: the whole genome sequences obtained were used to build a database and to perform multiple sequence alignment analysis; firstly, a sequence with high accuracy (high sequencing quality) of a bacterial whole genome sequence was used as the target sequence, and the whole genome sequences of other strains belonging to the same species of bacteria were compared with each other to obtain the consensus sequence between the two, and then multiple sequence alignment and comprehensive analysis were performed on the consensus sequences to obtain the overlap sequence, which was the intraspecific consensus sequence of the species;
3) Acquisition of inter-specie specific sequences: three BLAST comparisons of the obtained intraspecies consensus sequence of each bacterium with the intraspecies consensus sequence of all bacteria, all bacterial genome sequences (excluding self-alignment when comparing), and human genome (hg19) sequence were performed, then the non-overlapping sequences were used as interspecies specific sequences, which were the unique DNA sequences of pathogenic bacteria.
4) Removal of repetitive sequences: repetitive sequences were masked using tools such as RepeatMasker.

In this way, we obtained a plurality of specific nucleic acid fragments of a variety of bacteria, and their nucleotide sequences are set forth in SEQ ID No: 34-1590, respectively, in which each bacterium could have a plurality of specific nucleic acid fragments.

After the sequences of these pathogen specific nucleic acid fragments are obtained, they can be conveniently used to perform the detection of various pathogens in patients or patient samples. For example, the presence of a specific pathogen specific nucleic acid fragment could be used to indicate the inclusion of that particular pathogen in a patient or patient sample; the content of a particular pathogen specific nucleic acid fragment can be used to indicate the content of that particular pathogen in a patient or patient sample. The sequences of these pathogen specific nucleic acid fragments may themselves also be included in some assay kits, for example as positive controls. Understandably, during the assay, the full length of these specific nucleic acid fragments (SEQ ID No: 34-1590) provided herein can be detected; alternatively, only a part of its fragments, such as those of 50, 60, 80, 100 nucleotides or more in length, are detected. Similarly, the pathogen specific nucleic acid fragments included in the kit may also be full-length fragments or partial fragments (e.g. fragments of 50, 60, 80, 100 nucleotides or more in length). In some preferred embodiments, the length of the pathogen specific nucleic acid fragment is at least 60 nucleotides.

### Nucleic Acid Amplification

The application of nucleic acid amplification technology has changed the way of microbial pathogens diagnosis, and is also a common molecular biology technique used. In the 1980s, a variety of techniques for DNA amplification successively emerged, mainly including polymerase chain reaction (PCR) technique, ligase chain reaction (LCR) technique, as well as isothermal amplification.

The discovery and application of DNA polymerases with high-temperature resistance properties have made PCR the most commonly used DNA amplification technique. The principle of PCR technology to detect pathogenic microorganisms is to utilize specific oligonucleotide strands as primers and target nucleic acids containing the sequence to be amplified as templates to achieve exponential amplification of double stranded DNA by constantly switching the temperature, resulting in a large number of DNA fragments of interest (ie, amplification products) for subsequent identification. The advantages of this technique are its high sensitivity and easy operation, which can complete the detection of very trace amounts of pathogenic bacteria, which is essential for the detection of pathogenic microorganisms characterized by long growth cycles, harsh culture conditions or biochemical reactions.

LCR is another in vitro amplification technique developed after the advent of PCR technology. The principle of this technique is that: using a high temperature resistant DNA ligase and four primers, two adjacent forward primers and two reverse primers paired with their reverse complement. There is usually 1 gap between the two adjacent primers, which serves as a template for DNA ligase ligation. DNA ligase specificity is high and base mismatches are not tolerated, so this technique is frequently used for the detection of SNPs.

Isothermal amplification of nucleic acids is a facile technique that allows rapid and efficient accumulation of nucleic acid sequences at a constant temperature, and a variety of isothermal amplification techniques have been developed since the early 1990s as an alternative to PCR. In contrast to PCR techniques, isothermal amplification techniques, which do not require complicated thermal cycling processes (and therefore reduce the cost of amplification), enable amplification reactions at only one specific temperature. Isothermal amplification techniques commonly used in the art include: nucleic acid sequence based amplification (NASBA), strand displacement amplification (SDA), recombinase polymerase amplification (RPA), helicase dependent isothermal DNA amplification (HDA) Loop mediated isothermal amplification (lamp), rolling circle amplification (RCA), and others. These isothermal amplification techniques are well known to those skilled in the art and are not further described here.

In some embodiments herein, nucleic acids in a sample may be amplified prior to detection of pathogen specific nucleic acid fragments to increase the sensitivity of detection.

### CRISPR/Cas Genome Editing System and Trans Cleavage Activity of Cas12a

The CRISPR/Cas system has the potential for targeted cleavage of nucleic acid molecules, and genome editing tools based on this system are progressively being developed and utilized in significant quantities. Currently, genome editing tool applications based on CRISPR/Cas9 and CRISPR/Cas12a systems are the most prevalent. Their working principles are briefly described as follows: first, with the guidance of an RNA molecule, Cas proteins directionally cleave the double stranded DNA of a target gene, causing the integrity of the DNA strand to be disrupted; The corresponding DNA repair system within the cell is then activated, mainly including the NHEJ repair mechanism versus the HDR repair mechanism, thereby completing the destruction or directed remodeling of the target genes.

Compared with previous genome editing tools, CRISPR/Cas based genome editing technology has considerable advantages: it is simple in composition and consists of only one Cas protein along with sgRNA (for cas12a, only crRNA), so that editing targeting different loci requires only changing different sgRNAs (or just changing the seed region sequence in which the target nucleic acid binds). Functional studies of multiple genes can be made possible by designing multiple pairs of sgRNAs to achieve simultaneous editing of multiple gene loci.

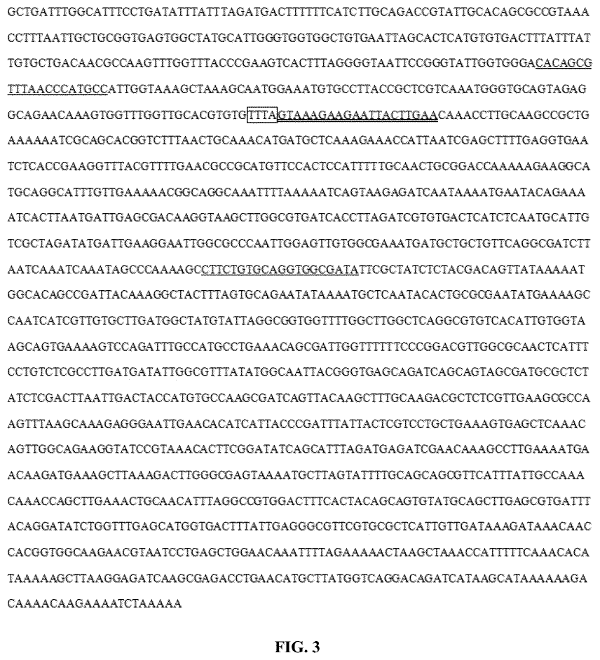

Recently, Cas12a was found to has not only cis cleavage activity for targeted cleavage of DNA, but also trans cleavage activity for nonspecific cleavage of arbitrary single stranded DNA (see FIG. 2). Upon binding to the target DNA with reverse complementary pairing of the crRNA, the Cas12a crRNA complex provokes Cas12a's trans cleavage activity, resulting in Cas12a indiscriminate cleavage of an arbitrary single stranded DNA molecule in the vicinity (i.e., without sequence specificity). Therefore, in the presence of a single stranded reporter DNA molecule, the trans cleavage activity of Cas12a can be exploited to detect the target DNA molecule. The detection process includes, for example: first utilizing exponential amplification of the target DNA in the sample to be tested by techniques such as PCR or RPA to increase the sensitivity of the assay; The amplified products are then examined using Cas12a, crRNA as well as single stranded reporter DNA, and if a DNA sequence corresponding to the seed region of crRNA is present in the amplification product (a PAM sequence such as ttta is also present 5′ upstream), the trans cleavage activity of Cas12a will be excited to cleave the reporter DNA, resulting in a fluorescent signal.

In some embodiments, the crRNA used in conjunction with Cas12crRNA consists of a 21-nt backbone and a 20- to 24-nt seed region/spacer for target sequence recognition. In a more specific embodiment, the sequence of the crRNA is: 5′-UAAUUUCUA CUAAGUGUAGAU (backbone region, SEQ ID No: 1591)+20-24 nt seed region-3′. In some embodiments, the corresponding crRNA can be transcribed in vitro with T7 RNA polymerase by using DNA as a template, and pure crRNA can be obtained by purification.

Single stranded reporter DNA molecules have a fluorescent group modification at one end and a quencher group modification at the other end. Due to the presence of the quencher group, the intact single stranded reporter DNA molecule does not produce a fluorescent signal. After cleaved by the trans cleavage activity of cas12a, the quencher group is separated from the fluorescent group, resulting in a fluorescent signal. The presence or intensity of the fluorescent signal is suggestive of the absence or amount of amplified product.

In this study, we first utilized bioinformatics methods to obtain intraspecific DNA sequences of several microorganisms, and specific crRNA for targeted identification of pathogenic bacteria were designed and synthesized according to these sequences. Next, we utilized Escherichia coli prokaryotic expression to purify lbCas12a protein derived from Lachnospiraceae bacterium, verifying that the protein possesses cis and trans cleavage activity. Subsequently, a CRISPR/Cas12a based assay for pathogenic bacteria was developed using single stranded reporter DNA, lbCas12a protein with specific crRNA, assisted by PCR amplification, and validated using 10 common severe pneumonia pathogens as an example and found to be successful in detecting these pathogens based on the specie-specific nucleic acid sequences of each pathogen, and its accuracy and specificity were verified. Ultimately, rapid detection of clinical samples from patients with severe pneumonia was achieved within 3 hours using CRISPR/Cas12a based pathogen detection tools. The CRISPR/Cas12a based detection method for pathogenic bacteria provided herein is expected to become a novel and rapid method for clinical pathogenic bacteria identification and provide an important technical support to improve the diagnosis and treatment of severe pneumonia.

In some embodiments herein, the trans cleavage activity of Cas12a is exploited to enable detection of pathogen specific nucleic acid fragments or their amplified products, which further increases the specificity of detection due to the requirement for complementary pairing of the crRNA with the target DNA.

the present invention will be further described in detail through specific embodiments.

### Example 1. Validation of the CRISPR/Cas Based Method for the Detection of Pathogenic Bacteria

The pathogenic bacteria used for the detection were all obtained from the intensive care unit of Drum Tower Hospital. The clinical samples were from patients with severe pneumonia, and were isolated and cultured by the Department of microbiological examination of Drum Tower Hospital to determine the strain species (each pathogenic bacterium had two strains, which were derived from different patients).

TABLE 1
the isolated and cultured strains of pathogenic bacteria

| NO. | Latin name | Short name |
|---|---|---|
| 1 | Acinetobacter baumannii | Aci_ba |
| 2 | Escherichia coli [symbol] | E. coli |
| 3 | Klebsiella pneumoniae | Kle_pn |
| 4 | Staphylococcus aureus | Sta_au |
| 5 | Pseudomonas aeruginosa | Pse_ae |
| 6 | Staphylococcus epidermidis | Sta_ep |
| 7 | Staphylococcus capitis | Sta_ca |
| 8 | Enterococcus faecalis | Ent_fa-is |
| 9 | Enterococcus faecium | Ent_fa-um |
| 10 | Stenotrophomonas maltophilia | Ste_ma |

### 1. Specificity Test

To demonstrate the specificity and reliability of the CRISPR/Cas system based assay for pathogenic bacteria, the primers for amplification and corresponding crRNA were tested for cross validation.

### 1.1 Primer Specificity Test

To validate the specificity of the amplification primers, each set of primers was used in a cross reaction with extracted genomic DNA from 10 bacteria.

The forward and reverse primers among the amplification primers used were:
SEQ ID No: 1 and 2 for amplification of genomic DNA from Acinetobacter baumannii;
SEQ ID No: 4 and 5 for amplification of genomic DNA from Klebsiella pneumoniae;
SEQ ID No: 7 and 8 for amplification of genomic DNA from Staphylococcus aureus;
SEQ ID No: 10 and 11 for amplification of genomic DNA from Escherichia coli;
SEQ ID No: 13 and 14 for amplification of genomic DNA from Pseudomonas aeruginosa;
SEQ ID No: 16 and 17 for amplification of genomic DNA from Stenotrophomonas maltophilia;
SEQ ID No: 19 and 20 for amplification of genomic DNA from Staphylococcus epidermidis;
SEQ ID No: 22 and 23 for amplification of genomic DNA from Enterococcus faecium;
SEQ ID No: 25 and 26 for amplification of genomic DNA from Staphylococcus capitis;
And SEQ ID No: 28 and 29 for amplification of genomic DNA from Enterococcus faecalis.

The target sequences (or seed regions) of crRNA used in combination with the above primer pairs were as set forth in SEQ ID No: 3, 6, 9, 12, 15, 18, 21, 24, 27, and 30.

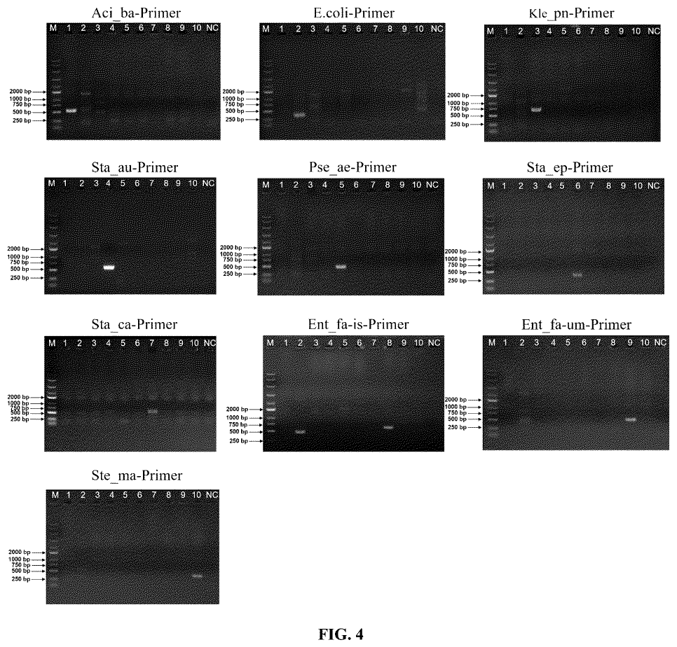

FIG. 3 shows an example of a specific pathogen specific nucleic acid fragment (from Acinetobacter baumannii, SEQ ID No: 36) to design amplification primers and crRNA sequences in which sequences corresponding to upstream primer and downstream primer (SEQ ID No: 1 and 2) are underlined, target sequence of crRNA (SEQ ID No: 3) is double underlined, and PAM sequence is boxed.

PCR was performed using I-5™ Master Mix (Tsingke). Since this polymerase readily produces nonspecific amplicons when amplifying fragments from Escherichia coli and Staphylococcus aureus DNA, we used 2×T5 Super PCR Mix (Tsingke) and TIANSeq HiFi Amplification Mix (Tiangen) for these two instead.

I-5™ Master Mix PCR reaction system:

| | | |
|---|---|---|
| 1-5 ™ 2 × High-Fidelity Master Mix | 25 | μl |
| Template | 2-10 | ng |
| Forward primer (10 μM) | 2 | μl |
| Reverse primer (10 μM) | 2 | μl |

| | |
|---|---|
| ddH₂O | to 50 μl |

2×T5 Super PCR Mix PCR reaction system:

| | | |
|---|---|---|
| 2 × T5 Super PCR Mix | 25 | μl |
| Template | 50-100 | ng |
| Forward primer (10 μM) | 2 | μl |
| Reverse primer (10 μM) | 2 | μl |

| | |
|---|---|
| ddH₂O | to 50 μl |

TIANSeq HiFi Amplification Mix PCR reaction system:

| | | |
|---|---|---|
| 2 × HiFi Amplification Mix | 25 | μl |
| Template | 10-50 | ng |
| Forward primer(10 μM) | 2 | μl |
| Reverse primer(10 μM) | 2 | μl |

| | |
|---|---|
| ddH₂O | to 50 μl |

PCR reaction system:

| | | | | |
|---|---|---|---|---|
| 98° C. | 2 | min | | |
| 98° C. | 10 | s | | |
| 52° C. | 10 | s | {close oversize brace} | 35 cycles |
| 72° C. | 15 | s | | |
| 72° C. | 5 | min | | |

| | |
|---|---|
| 4° C. | Pause |

Corresponding negative controls were set up with each set of primers, in which water was used as template.

After the PCR reaction was completed, products were mixed with corresponding volume of 6× Gel loading dye (NEB) and separated by agarose electrophoresis. Primer specificity was validated according to the electrophoresis results with or without a distinct bright band of the target DNA sizes.

1.2 crRNA Specificity Test

The PCR products amplified by the corresponding primers were detected using lbCas12a and the above crRNA designed according to the specie-specific DNA sequence of each pathogen. Three replicates were set up for each sample.

### Reaction System:

| | | |
|---|---|---|
| ssDNA-reporter (10 μM) | 1 | μl |
| PCR products/unpurified | 1-2 | μl |
| PCR reaction solution | | |
| 10 × NEBuffer ™ 3.1 | 2 | μl |
| lbCas12a | 1300 | ng |
| crRNA | 180 | ng |

| | |
|---|---|
| ddH₂O | to 20 μl |

in which the structure of the ssDNA reporter is: 5′-FAM-TTATT-BHQ1-3′.

The above reaction was added to a 384-well plate and incubated for 30˜45 min at 37° C. After the reaction, the fluorescence value of each well was detected using a microplate reader (infinite M200 Pro multi-purpose microplate reader, Austria TECAN) with the following detection parameters set:

| Excitation Wavelength | 485 | nm |
|---|---|---|
| Emission Wavelength | 535 | nm |
| Excitation Bandwidt | 9 | nm |
| Emission Bandwidth | 20 | nm |
| Gain | 100 | Manual |
| Number of Flashes | 25 | |
| Integration Time | 20 | μs |
| Lag Time | 0 | μs |
| Settle Time | 0 | ms |
| Z-Position (Manual) | 20000 | μm |

### 2. Experimental Results

### 2.1 Results of Primer Specificity Validation Test

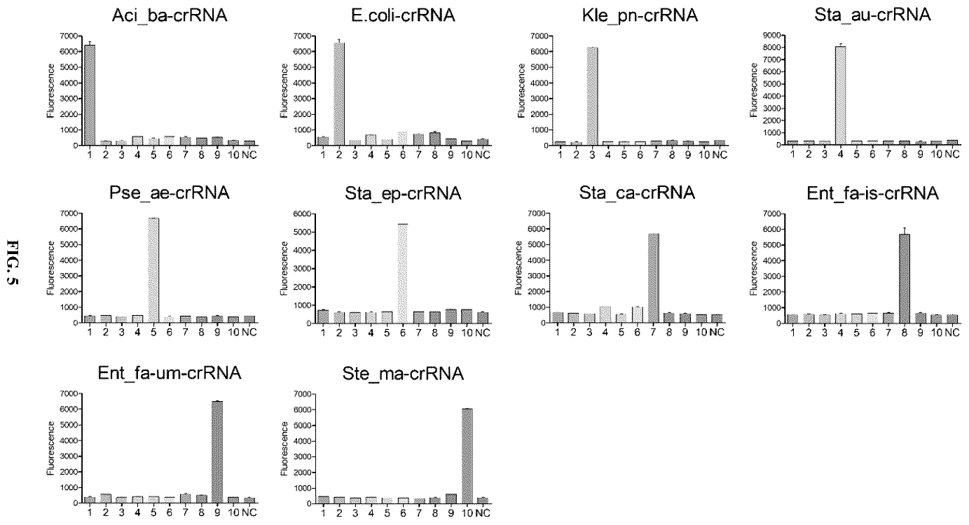

As shown in FIG. 4, although there were individual nonspecific bands, a large number of DNA products of interest could be amplified when the corresponding pathogenic bacteria genomic DNA was used as the template, illustrating the good specificity of primers for 10 pathogenic bacteria.

2.2 Results of crRNA Specificity Validation Test

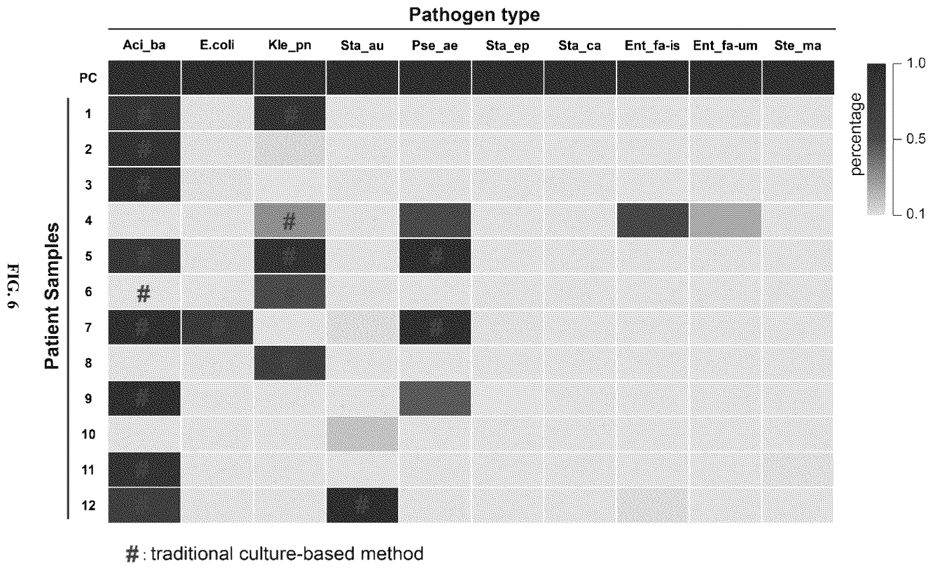

The PCR reaction liquid after amplification with the above corresponding primers was tested using lbCas12a along with the crRNA corresponding to each pathogenic bacterium. The fluorescence results in FIG. 5 show that the crRNA of each pathogenic bacterium can only show obvious fluorescent signals when detecting the PCR products amplified with this pathogenic bacterium as a template and the corresponding primers, indicating that the combination with crRNA makes the specificity of this detection method excellent.

### Example 2. Detection of Clinical Samples from Patients with Severe Pneumonia Using CRISPR/Cas System

### 1. Experimental Protocol

The 12 clinical samples (sputum or alveolar lavage fluid) were all obtained from the intensive care unit of Drum Tower Hospital and contrasted with the types of pathogenic bacteria identified by the Department of Microbiology Laboratory of Drum Tower Hospital using traditional culture assays.

1.1 DNA Extraction from Clinical Samples by the Kit

The extraction of DNA from clinical samples was performed with reference to the Quick-DNA/RNA™ Pathogen Miniprep Kit ((ZYMO RESEARCH) according to the manual introduction:
a) 50-200 μl of sample were pipetted, and 800 μl DNA/RNA shield reagent was added, followed by vortex for 60 s.
b) The sample was centrifuged at 16,000×g for 1 min and 200 μl supernatant was collected.
c) 2.0 μl proteinase K reagent were added to 200 μl of the supernatant, and mixed well.
d) 1 ml of pathogen DNA/RNA buffer reagent were added, mixed and then incubated for 5 min at room temperature.
e) The above solution was pipetted to the DNA binding column (which had been placed in a recovery tube), centrifuged for 30 s, and the solution passing through the column was discarded.
f) 500 μl of pathogen DNA/RNA wash buffer were added to the column, centrifuged for 30 s, and the solution passing through the column was discarded. This step was repeated once.
g) 500 μl ethanol (95-100%) were added to the column, centrifuged at 16,000×g for 1 min to ensure ethanol was eliminated completely. The recovery tube was discarded and the DNA binding column was placed in a DNase free 1.5 ml centrifuge tube.
h) 50 μl of double-distilled water at 65° C. were pipetted into the matrix of the column, stood alone for 2˜5 min, centrifuged at 16,000×g for 1 min, then the eluate was collected.
i) The DNA concentration was determined, and the sample was stored at −20° C. freezer.

### 1.2 PCR Reaction to Amplify Target Sequences

Using extracted total DNA from clinical samples as template, each of them was amplified using the 10 primer pairs described in Example 1. For each pair of primers, the template used for the positive control group was the corresponding genomic DNA of pathogenic bacteria, and the template for the negative control group was water.

The PCR reaction system was the same as demonstrated in Example 1.

### 1.3 Cas12a Detection

Unpurified PCR reaction solution was detected using lbCas12a, crRNA and ssDNA reporter. For the same sample to be tested, 3 replicates were set up; The detection reaction system and reaction conditions were the same as demonstrated in Example 1.

### 2. Experimental Results

PCR amplification was performed on total DNA from 12 clinical samples of patients with severe pneumonia using 10 pairs of amplification primers, followed by detection of the corresponding PCR reaction using single stranded reporter DNA, lbCas12a and relative crRNA for 10 pathogens. The fluorescence signal heat map is shown in FIG. 6. From the figure, the pathogenic bacteria based on CRISPR/Cas12a detection for clinical sample No. 1 were Acinetobacter baumannii and Klebsiella pneumoniae, those in clinical samples No. 2, 3, and 11 were Acinetobacter baumannii, those in clinical sample No. 4 were Klebsiella pneumoniae, Enterococcus faecalis, Enterococcus faecium and Pseudomonas aeruginosa, those in clinical sample No. 5 were Acinetobacter baumannii, Pseudomonas aeruginosa and Klebsiella pneumoniae, those in clinical samples No. 6 and 8 were Klebsiella pneumoniae, those in clinical sample No. 7 were Acinetobacter baumannii, Escherichia coli and Pseudomonas aeruginosa, those in clinical sample No. 9 were Acinetobacter baumannii and Pseudomonas aeruginosa, clinical sample No. 10 had a weak fluorescent signal when tested for Staphylococcus aureus and may have a small content of Staphylococcus aureus, and those in sample 12 were Acinetobacter baumannii and Staphylococcus aureus.

When compared with the traditional assay based on the culture of isolated colonies, 8 out of 12 samples showed consistent results. The differences were the detection of Pseudomonas aeruginosa, Enterococcus faecalis and Enterococcus faecium in clinical sample No. 4, the absence of Acinetobacter baumannii in clinical sample No. 6, the detection of Pseudomonas aeruginosa in clinical sample No. 9 and a small amount of Staphylococcus aureus in clinical sample No. 10, which was later shown to have a small amount of Staphylococcus aureus in clinical sample No. 10 by genetic sequencing.

In conclusion, the developed CRISPR/cas12a based pathogenic bacteria detection tool in this study exhibited a very high positive detection rate (11/12, 91.67%) compared with traditional detection methods that rely on the isolation and culture of pathogenic bacteria, which shortened the traditional detection cycle from several days to less than 3 hours, providing preliminary evidence that this assay can be used as a potential test for rapid diagnosis of severe pneumonia pathogenic bacteria types. In addition, based on the specie-specific nucleic acid fragments of numerous pathogens presented herein, similar approaches can be employed for the identification of a variety of other infectious diseases other than pneumonia associated pathogens.

1: A method for identifying one or more pathogens, comprising:
1) providing a sample that may comprise the pathogen; and
2) detecting presence or absence of a pathogen specific nucleic acid fragment in the sample or detecting content of a pathogen specific nucleic acid fragment in the sample,
wherein a pathogen specific nucleic acid fragment corresponding to Acinetobacter baumannii is selected from at least a part of any sequence of SEQ ID No: 34-49, or a complementary sequence thereof;
a pathogen specific nucleic acid fragment corresponding to Escherichia coli is selected from at least a part of any sequence of SEQ ID No: 50-221, or a complementary sequence thereof;
a pathogen specific nucleic acid fragment corresponding to Klebsiella pneumoniae is selected from at least a part of any sequence of SEQ ID No: 222-542, or a complementary sequence thereof;
a pathogen specific nucleic acid fragment corresponding to Staphylococcus aureus is selected from at least a part of any sequence of SEQ ID No: 543-601, or a complementary sequence thereof;
a pathogen specific nucleic acid fragment corresponding to Pseudomonas aeruginosa is selected from at least a part of any sequence of SEQ ID No: 602-896, or a complementary sequence thereof;
a pathogen specific nucleic acid fragment corresponding to Staphylococcus epidermidis is selected from at least a part of any sequence of SEQ ID No: 897-1079, or a complementary sequence thereof;
a pathogen specific nucleic acid fragment corresponding to Staphylococcus capitis is selected from at least a part of any sequence of SEQ ID No: 1080-1169, or a complementary sequence thereof;
a pathogen specific nucleic acid fragment corresponding to Enterococcus faecalis is selected from at least a part of any sequence of SEQ ID No: 1170-1279, or a complementary sequence thereof;
a pathogen specific nucleic acid fragment corresponding to Enterococcus faecium is selected from at least a part of any sequence of SEQ ID No: 1280-1405, or a complementary sequence thereof;
a pathogen specific nucleic acid fragment corresponding to Stenotrophomonas maltophilia is selected from at least a part of any sequence of SEQ ID No: 1406-1550, or a complementary sequence thereof;
a pathogen specific nucleic acid fragment corresponding to Mycobacterium tuberculosis, Mycobacterium africanum, or Mycobacterium bovis is selected from at least a part of any sequence of SEQ ID No: 1551-1590, or a complementary sequence thereof,
wherein the presence or content of the pathogen specific nucleic acid fragment reflects the presence or content of the pathogen corresponding to the pathogen-specific nucleic acid fragment in the sample, respectively.

2: The method according to claim 1, wherein step (2) further comprises performing extraction of nucleic acid from the sample prior to the detection.

3: The method according to claim 1, wherein step (2) comprises performing an amplification reaction using the pathogen specific nucleic acid fragment in the sample as a template, and determining the presence or content of the pathogen-specific nucleic acid fragment by detecting presence or content of amplified product.

4: The method according to claim 1, wherein the amplification reaction is Polymerase Chain Reaction (PCR).

5: The method according to claim 1, wherein in the PCR:
primers used for amplification of pathogen specific nucleic acid fragment of Acinetobacter baumannii comprise sequences as set forth in SEQ ID No: 1 and SEQ ID No: 2;
primers used for amplification of pathogen specific nucleic acid fragment of Klebsiella pneumoniae comprise sequences as set forth in SEQ ID No: 4 and SEQ ID No: 5;
primers used for amplification of pathogen specific nucleic acid fragment of Staphylococcus aureus comprise sequences as set forth in SEQ ID No: 7 and SEQ ID No: 8;
primers used for amplification of pathogen specific nucleic acid fragment of Escherichia coli comprise sequences as set forth in SEQ ID No: 10 and SEQ ID No: 11;
primers used for amplification of pathogen specific nucleic acid fragment of Pseudomonas aeruginosa comprise sequences as set forth in SEQ ID No: 13 and SEQ ID No: 14;
primers used for amplification of pathogen specific nucleic acid fragment of Stenotrophomonas maltophilia comprise sequences as set forth in SEQ ID No: 16 and SEQ ID No: 17;
primers used for amplification of pathogen specific nucleic acid fragment of Staphylococcus epidermidis comprise sequences as set forth in SEQ ID No: 19 and SEQ ID No: 20;
primers used for amplification of pathogen specific nucleic acid fragment of Enterococcus faecium comprise sequences as set forth in SEQ ID No: 22 and SEQ ID No: 23;
primers used for amplification of pathogen specific nucleic acid fragment of Staphylococcus capitis comprise sequences as set forth in SEQ ID No: 25 and SEQ ID No: 26;
primers used for amplification of pathogen specific nucleic acid fragment of Enterococcus faecalis comprise sequences as set forth in SEQ ID No: 28 and SEQ ID No: 29;
primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32.

6: The method according to claim 1, wherein the detection of amplification product is performed utilizing trans cleavage activity of a nuclease of CRISPR/Cas family.

7: The method according to claim 1, wherein the nuclease of CRISPR/Cas family is Cas12.

8: The method according to claim 1, wherein the nuclease of CRISPR/Cas family is lbCas12.

9: The method according to claim 1, wherein:
primers used for amplification of pathogen specific nucleic acid fragment of Acinetobacter baumannii comprise sequences as set forth in SEQ ID No: 1 and SEQ ID No: 2, and a target sequence of crRNA used in combination with the nuclease of CRISPR/Cas family comprises a sequence as set forth in SEQ ID No: 3;
primers used for amplification of pathogen specific nucleic acid fragment of Klebsiella pneumoniae comprise sequences as set forth in SEQ ID No: 4 and SEQ ID No: 5, and a target sequence of crRNA used in combination with the nuclease of CRISPR/Cas family comprises a sequence as set forth in SEQ ID No: 6;
primers used for amplification of pathogen specific nucleic acid fragment of Staphylococcus aureus comprise sequences as set forth in SEQ ID No: 7 and SEQ ID No: 8, and a target sequence of crRNA used in combination with the nuclease of CRISPR/Cas family comprises a sequence as set forth in SEQ ID No: 9;
primers used for amplification of pathogen specific nucleic acid fragment of Escherichia coli comprise sequences as set forth in SEQ ID No: 10 and SEQ ID No: 11, and a target sequence of crRNA used in combination with the nuclease of CRISPR/Cas family comprises a sequence as set forth in SEQ ID No: 12;
primers used for amplification of pathogen specific nucleic acid fragment of Pseudomonas aeruginosa comprise sequences as set forth in SEQ ID No: 13 and SEQ ID No: 14, and a target sequence of crRNA used in combination with the nuclease of CRISPR/Cas family comprises a sequence as set forth in SEQ ID No: 15;
primers used for amplification of pathogen specific nucleic acid fragment of Stenotrophomonas maltophilia comprise sequences as set forth in SEQ ID No: 16 and SEQ ID No: 17, and a target sequence of crRNA used in combination with the nuclease of CRISPR/Cas family comprises a sequence as set forth in SEQ ID No: 18;
primers used for amplification of pathogen specific nucleic acid fragment of Staphylococcus epidermidis comprise sequences as set forth in SEQ ID No: 19 and SEQ ID No: 20, and a target sequence of crRNA used in combination with the nuclease of CRISPR/Cas family comprises a sequence as set forth in SEQ ID No: 21;
primers used for amplification of pathogen specific nucleic acid fragment of Enterococcus faecium comprise sequences as set forth in SEQ ID No: 22 and SEQ ID No: 23, and a target sequence of crRNA used in combination with the nuclease of CRISPR/Cas family comprises a sequence as set forth in SEQ ID No: 24;
primers used for amplification of pathogen specific nucleic acid fragment of Staphylococcus capitis comprise sequences as set forth in SEQ ID No: 25 and SEQ ID No: 26, and a target sequence of crRNA used in combination with the nuclease of CRISPR/Cas family comprises a sequence as set forth in SEQ ID No: 27;
primers used for amplification of pathogen specific nucleic acid fragment of Enterococcus faecalis comprise sequences as set forth in SEQ ID No: 28 and SEQ ID No: 29, and a target sequence of crRNA used in combination with the nuclease of CRISPR/Cas family comprises a sequence as set forth in SEQ ID No: 30;
primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32, and a target sequence of crRNA used in combination with the nuclease of CRISPR/Cas family comprises a sequence as set forth in SEQ ID No: 33.

10: The method according to claim 1, wherein the sample is sputum or alveolar lavage fluid from a patient with severe pneumonia.

11: The method according to claim 1, wherein identification is performed in step 2) for a plurality of pathogens, including Acinetobacter baumannii, Escherichia coli, Klebsiella pneumoniae, Staphylococcus aureus, Pseudomonas aeruginosa, Staphylococcus epidermidis, Staphylococcus capitis, Enterococcus faecalis, Enterococcus faecium, and Stenotrophomonas maltophilia.

12: The method according to claim 1, wherein the plurality of pathogens further comprise Mycobacterium tuberculosis, Mycobacterium africanum, or d Mycobacterium bovis.

13: An isolated nucleic acid molecule comprising at least a part of any sequence of SEQ ID No: 34-1590, or a complementary sequence thereof.

14: The nucleic acid molecule according to claim 13, having a length of not less than 60 nucleotides.

15: A kit for detection of pathogen in a sample, comprising:
1) primers for amplification of a pathogen specific nucleic acid fragments in the sample to generate amplification product; and
2) a nuclease of CRISPR/Cas family with trans cleavage activity, a crRNA using at least a part of sequence of the amplification product as target sequence, and a single-stranded DNA reporter molecule with a fluorescent group and a quencher group at the 5′ and 3′ ends, respectively, wherein
a pathogen specific nucleic acid fragment corresponding to Acinetobacter baumannii is selected from at least a part of any sequence of SEQ ID No: 34-49, or a complementary sequence thereof;
a pathogen specific nucleic acid fragment corresponding to Escherichia col is selected from at least a part of any sequence of SEQ ID No: 50-221, or a complementary sequence thereof;
a pathogen specific nucleic acid fragment corresponding to Klebsiella pneumoniae is selected from at least a part of any sequence of SEQ ID No: 222-542, or a complementary sequence thereof;
a pathogen specific nucleic acid fragment corresponding to Staphylococcus aureus is selected from at least a part of any sequence of SEQ ID No: 543-601, or a complementary sequence thereof;
a pathogen specific nucleic acid fragment corresponding to Pseudomonas aeruginosa is selected from at least a part of any sequence of SEQ ID No: 602-896, or a complementary sequence thereof;
a pathogen specific nucleic acid fragment corresponding to Staphylococcus epidermidis is selected from at least a part of any sequence of SEQ ID No: 897-1079, or a complementary sequence thereof;
a pathogen specific nucleic acid fragment corresponding to Staphylococcus capitis is selected from at least a part of any sequence of SEQ ID No: 1080-1169, or a complementary sequence thereof;
a pathogen specific nucleic acid fragment corresponding to Enterococcus faecalis is selected from at least a part of any sequence of SEQ ID No: 1170-1279, or a complementary sequence thereof;
a pathogen specific nucleic acid fragment corresponding to Enterococcus faecium is selected from at least a part of any sequence of SEQ ID No: 1280-1405, or a complementary sequence thereof;
a pathogen specific nucleic acid fragment corresponding to Stenotrophomonas maltophilia is selected from at least a part of any sequence of SEQ ID No: 1406-1550, or a complementary sequence thereof; and
a pathogen specific nucleic acid fragment corresponding to Mycobacterium tuberculosis, Mycobacterium africanum, or Mycobacterium bovis is selected from at least a part of any sequence of SEQ ID No: 1551-1590, or a complementary sequence thereof.

16: The kit according to claim 15, further comprising an isolated nucleic acid molecule comprising at least a part of any sequence of SEQ ID No: 34-1590, or a complementary sequence thereof as a positive control.

17: The kit according to claim 15, wherein the nuclease of CRISPR/Cas family is lbCas12.

18: The kit according to claim 15, wherein:
the primers used for amplification of pathogen specific nucleic acid fragment of Acinetobacter baumannii comprise sequences as set forth in SEQ ID No: 1 and SEQ ID No: 2;
the primers used for amplification of pathogen specific nucleic acid fragment of Klebsiella pneumoniae comprise sequences as set forth in SEQ ID No: 4 and SEQ ID No: 5;
the primers used for amplification of pathogen specific nucleic acid fragment of Staphylococcus aureus comprise sequences as set forth in SEQ ID No: 7 and SEQ ID No: 8;
the primers used for amplification of pathogen specific nucleic acid fragment of Escherichia coli comprise sequences as set forth in SEQ ID No: 10 and SEQ ID No: 11;
the primers used for amplification of pathogen specific nucleic acid fragment of Pseudomonas aeruginosa comprise sequences as set forth in SEQ ID No: 13 and SEQ ID No: 14;
the primers used for amplification of pathogen specific nucleic acid fragment of Stenotrophomonas maltophilia comprise sequences as set forth in SEQ ID No: 16 and SEQ ID No: 17;
the primers used for amplification of pathogen specific nucleic acid fragment of Staphylococcus epidermidis comprise sequences as set forth in SEQ ID No: 19 and SEQ ID No: 20;
the primers used for amplification of pathogen specific nucleic acid fragment of Enterococcus faecium comprise sequences as set forth in SEQ ID No: 22 and SEQ ID No: 23;
the primers used for amplification of pathogen specific nucleic acid fragment of Staphylococcus capitis comprise sequences as set forth in SEQ ID No: 25 and SEQ ID No: 26;
the primers used for amplification of pathogen specific nucleic acid fragment of Enterococcus faecalis comprise sequences as set forth in SEQ ID No: 28 and SEQ ID No: 29;
the primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32.

19: The kit according to claim 15, wherein:
the primers used for amplification of pathogen specific nucleic acid fragment of Acinetobacter baumannii comprise sequences as set forth in SEQ ID No: 1 and SEQ ID No: 2, and a target sequence of the crRNA comprises a sequence as set forth in SEQ ID No: 3;
the primers used for amplification of pathogen specific nucleic acid fragment of Klebsiella pneumoniae comprise sequences as set forth in SEQ ID No: 4 and SEQ ID No: 5, and a target sequence of the crRNA comprises a sequence as set forth in SEQ ID No: 6;
the primers used for amplification of pathogen specific nucleic acid fragment of Staphylococcus aureus comprise sequences as set forth in SEQ ID No: 7 and SEQ ID No: 8, and a target sequence of the crRNA comprises a sequence as set forth in SEQ ID No: 9;
the primers used for amplification of pathogen specific nucleic acid fragment of Escherichia coli comprise sequences as set forth in SEQ ID No: 10 and SEQ ID No: 11, and a target sequence of the crRNA comprises a sequence as set forth in SEQ ID No: 12;
the primers used for amplification of pathogen specific nucleic acid fragment of Pseudomonas aeruginosa comprise sequences as set forth in SEQ ID No: 13 and SEQ ID No: 14, and a target sequence of the crRNA comprises a sequence as set forth in SEQ ID No: 15;
the primers used for amplification of pathogen specific nucleic acid fragment of Stenotrophomonas maltophilia comprise sequences as set forth in SEQ ID No: 16 and SEQ ID No: 17, and a target sequence of the crRNA comprises a sequence as set forth in SEQ ID No: 18;
the primers used for amplification of pathogen specific nucleic acid fragment of Staphylococcus epidermidis comprise sequences as set forth in SEQ ID No: 19 and SEQ ID No: 20, and a target sequence of the crRNA comprises a sequence as set forth in SEQ ID No: 21;
the primers used for amplification of pathogen specific nucleic acid fragment of Enterococcus faecium comprise sequences as set forth in SEQ ID No: 22 and SEQ ID No: 23, and a target sequence of the crRNA comprises a sequence as set forth in SEQ ID No: 24;
the primers used for amplification of pathogen specific nucleic acid fragment of Staphylococcus capitis comprise sequences as set forth in SEQ ID No: 25 and SEQ ID No: 26, and a target sequence of the crRNA comprises a sequence as set forth in SEQ ID No: 27;
the primers used for amplification of pathogen specific nucleic acid fragment of Enterococcus faecalis comprise sequences as set forth in SEQ ID No: 28 and SEQ ID No: 29, and a target sequence of the crRNA comprises a sequence as set forth in SEQ ID No: 30;
the primers used for amplification of pathogen specific nucleic acid fragment of Mycobacterium tuberculosis, Mycobacterium africanum, or Mycobacterium bovis comprise sequences as set forth in SEQ ID No: 31 and SEQ ID No: 32, and a target sequence of the crRNA comprises a sequence as set forth in SEQ ID No: 33.

20: The kit according to claim 15, wherein the sample is sputum or alveolar lavage fluid from a patient with severe pneumonia.

21-22. (canceled)
